(19) United States
(12) Patent Application Publication
(10) Pub. No.: US 2003/0219380 A1
(43) Pub. Date: Nov. 27, 2003

(54) METHOD OF DETERMINING AN EFFICACIOUS DOSE OF A DRUG

(76) Inventors: ANNIE FONG, SUNNYVALE, CA (US); KLAUS PETER HIRTH, SAN FRANCISCO, CA (US)

Correspondence Address: BETH A. BURROUS, FOLEY & LARDNER WASHINGTON HARBOUR, 3000 K STREET, N.W., SUITE 500, WASHINGTON, DC, 20007-5109, US

(21) Appl. No.: 09/186,475

(22) Filed: Nov. 4, 1998

Related U.S. Application Data
(60) Provisional application No. 60/064,935, filed on Nov. 7, 1997.

(51) Int. Cl. A61K 49/00; C12Q 1/68; G01N 33/574; A61K 31/404

(52) U.S. Cl. 424/9.1; 435/6; 435/7.23; 514/414

(57) ABSTRACT

The present invention relates to a method of determining an efficacious dose of a drug administered to a subject for the purpose of modulating angiogenesis, including conditions manifested by cell proliferation, cell differentiation, or cell survival. The method comprises the step of monitoring a marker related to angiogenesis. The drug used in the present invention is an indolinone compound. The invention also features a kit for determining the efficacious dose of the drug.

### RELATED APPLICATION

The present application claims priority to the U.S. provisional application, Serial No. 60/064,935, filed Nov. 7, 1997, by Fong, et al., and entitled “METHOD OF DETERMINING AN EFFICACIOUS DOSE OF A DRUG” (Lyon & Lyon Docket No. 227/174), which is hereby incorporated herein by reference in its entirety, including any drawings.

### INTRODUCTION

The present invention relates in part to methods of determining an efficacious dose of a drug for the purpose of modulating angiogenesis.

### BACKGROUND

The following description of the background of the invention is provided to aid in understanding the invention, but is not admitted to describe or constitute prior art to the invention.

The growth and formation of blood vessels occurs by a process termed “angiogenesis”. Many diseases are the result of, or involve, aberrant angiogenic activities. For example, in order for a cancerous tumor to develop, it may induce the surrounding tissue to provide a nutrient supply, and it does so by inducing angiogenesis (Denekamp, J., The British Journal of Radiology 66:181, 1993).

Current methods of treating cancers focus on the different rates of proliferation between normal and cancerous tissues. Cytotoxic drugs preferentially cause cell death in tissues that are dividing rapidly. To determine an effective dose of these types of drugs, the dose selection is often guided by measuring the maximum tolerated dose as determined by toxic side-effects to the patient.

### SUMMARY OF THE INVENTION

The present invention is directed in part to a method for determining an efficacious dose of a drug administered to a subject. By monitoring a marker involved in the process that the drug is intended to regulate, one may determine the effective dose of that drug.

For example, it would be difficult to directly monitor the effect of a drug on angiogenesis by directly measuring the inhibition of blood vessel formation. The method of the present invention enables the determination of an effective dose of a drug administered for the purpose of modulating angiogenesis by monitoring a surrogate marker.

There are certain advantages in practicing the present invention. For example, one may measure the effective dose of a drug without harming the patient with an overly toxic dose. Also, one can optimize the effect of the drug on a tumor while decreasing the undesirable side effects on the patient.

Thus in a first aspect the invention features a method for determining an efficacious dose of a drug administered to a subject for the purpose of modulating angiogenesis, comprising the step of monitoring a marker related to angiogenesis.

The term “efficacious”, as used herein, refers to a dose that brings about, accomplishes, or affects the purpose of administering such a dose, which could be, for example, modulating a relevant biological response of the tumor, such as its rate of growth.

The term “dose”, as used herein, refers to the unit amount of the drug that is administered. For treatment, the dose to be administered can depend on several factors, including the nature and activity of the drug to be administered, the manner in which the drug is administered, the abnormal condition to be treated, and the condition of the subject to which the drug is administered.

The term “drug”, as used herein, refers to a compound administered to potentially bring about a therapeutic effect. The compounds, however, may not, in fact, result in a therapeutic effect following administration.

The term “administered”, as used herein, refers to the provision of a compound. The term is understood to include broadly the provision of the compound to a subject, and more specifically, to a method of incorporating a compound into cells or tissues of a subject. The compound can be provided to cells or tissues of an organism which exist either within the subject or outside of the subject. Cells existing outside the subject can be maintained or grown in cell culture dishes. For cells harbored within the subject, many techniques exist in the art to administer compounds. Suitable routes of administration may, for example, include (but not be limited to) oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. For cells outside of the organism, multiple techniques exist in the art to administer the compounds, including (but not limited to) cell microinjection techniques, transformation techniques, and carrier techniques. All of these administration techniques are well-known to a person of ordinary skill in the art. Compounds of the invention can be administered periodically to a patient or can be administered continuously. The compounds of the invention can be administered once or in multiple doses to the patient.

The term “subject”, as used herein, refers to humans and other mammals. The subject refers preferably to such organisms as cows, mice, rats, rabbits, guinea pigs, and goats, more preferably to cats, dogs, monkeys, and apes, and most preferably to humans.

The phrase “modulating angiogenesis” refers to the activation or inhibition of angiogenesis.

The term “angiogenesis” refers to the general process by which blood vessels are formed.

The term “monitoring”, as used herein, refers to the process of checking, testing, keeping track of, regulating, or controlling.

The term “marker” refers to a means by which the angiogenic status of a subject is monitored. A marker can be a biological molecule that undergoes a determinable change related to, or possibly as a result of, the condition of interest. In the present invention, the condition of interest can be related to angiogenesis. Examples of markers include, but are in no way limited to, tissue factor, CD40, u-PA, ETS-1, IL8, and t-PA.

The term “sample” refers to a cell, tissue, organ, fluid, or any portion thereof, that is isolated from the body of the subject. Examples of a sample include, but are not limited to, a histologic section, a specimen obtained by biopsy, or cells that are placed in or adapted to tissue culture. If desired, a sample can be processed, for example, by homogenization, lysing, freezing, heating, fractionation, or various other processes well-known in the art.

In a preferred embodiment, the invention provides a method for determining an efficacious dose of a drug administered to a subject for the purpose of modulating angiogenesis, where the angiogenesis is modulated to treat or prevent conditions associated with angiogenesis including conditions manifested by cell proliferation, cell differentiation, or cell survival.

The term “cell proliferation” refers to the growth of cells. The term “cell death” refers to the unscheduled or premature death of a cell. Unscheduled or premature death is determined by comparison to otherwise normal, untreated, similarly situated cells. Cell proliferative conditions include, but are not limited to, aberrant cell proliferative conditions (cancers) such as fibrotic and mesangial disorders, abnormal angiogenesis and vasculogenesis, abnormal wound healing, psoriasis, diabetes mellitus, arthritis, diabetic retinopathy, and inflammation.

The term “cell differentiation” refers to the process by which cells mature and become less pluripotent, as that term is used in the art. The term also refers to a change of appearance or phenotype as compared to the average subject. Cell differentiation conditions include, but are not limited to, neurodegenerative disorders, slow wound healing rates, morphogenesis, and tissue grafting techniques.

The term “cell survival” refers to the unscheduled survival of a cell. Unscheduled or prolonged survival is determined by comparison to otherwise normal, untreated, similarly situated cells. Cell survival conditions relate to conditions in which programmed cell death (apoptosis) pathways are activated or abrogated. A number of protein kinases are associated with the apoptosis pathways. Aberrations in the function of any one of the protein kinases could lead to cell immortality or premature cell death.

Cell proliferation, differentiation, and survival are phenomena simply measured by methods well-known in the art. These methods can involve observing the number of cells or the appearance of cells under a microscope with respect to time (for example, days).

In a preferred embodiment, the cell proliferative disease is selected from the group consisting of cancer, arthritis, endometriosis, and diabetic retinopathy. These conditions are described hereinafter more fully. The invention also provides a method for determining an efficacious dose of a drug administered to a subject for the purpose of modulating angiogenesis, where the drug is administered at various time intervals or continuously and at various doses. The drug is selected from the group consisting of a preceptor agonist and a receptor antagonist in preferred embodiments.

The term “receptor” refers to a biological molecule that physically interacts with a substance, usually a ligand for the receptor, whereby, through a change in the physiology of the receptor, a signal is transduced by the receptor leading to a change in the biological function of the cell, such as mitogenesis, growth, or differentiation. Common receptor types include, but are not limited to, the growth hormone receptors, tyrosine kinase receptors, neurotransmitter receptors, and steroid hormone receptors.

The term “agonist”, as used herein, refers to a substance that acts positively on another substance. For example, an agonist will enhance phosphorylation by a protein kinase or interaction between a protein and its natural binding partner.

The term “antagonist”, as used herein, refers to a substance that acts negatively on another substance. For example, an antagonist will inhibit phosphorylation by a protein kinase or interaction between a protein and its natural binding partner.

The terms “treatment” and “treat”, as used herein, refer to the creation of a therapeutic effect and at least partially alleviating or abrogating the abnormal condition in the subject, or modulating a normal condition in the subject.

The term “therapeutic effect” refers to the inhibition of cell growth causing or contributing to an abnormal condition (e.g. cancer). The term “therapeutic effect” also refers to the inhibition of growth factors causing or contributing to the abnormal condition. A therapeutic effect relieves to some extent one or more of the symptoms of the abnormal condition. In reference to the treatment of a cancer, a therapeutic effect can, for example, refer to one or more of the following: (a) a reduction in tumor size; (b) inhibition (i.e., slowing or stopping) of tumor metastasis; (c) inhibition of tumor growth; and (d) relieving to some extent one or more of the symptoms associated with the abnormal condition. Compounds demonstrating efficacy against leukemias can be identified as described herein. These compounds may slow or decrease the invasiveness of cancer cells and thereby inhibit the progression of the disease.

The term “prevent”, as used herein, refers to decreasing the probability, or eliminating the possibility, that a subject contracts or develops the normal or abnormal condition.

In a preferred embodiment, the invention provides a method for determining an efficacious dose of a drug administered to a subject for the purpose of modulating angiogenesis, where the drug is a receptor antagonist that inhibits a receptor involved in angiogenesis. The receptor involved in angiogenesis is selected from the group consisting of Flt-1 and Flk-1. The drug is preferably an indolinone compound.

The term “indolinone” is used as that term is commonly understood in the art and includes a large subclass of substituted or unsubstituted compounds that are capable of being synthesized from an aldehyde moiety and an oxindole moiety.

The indolinone compounds of the invention have the structure set forth in formula I:

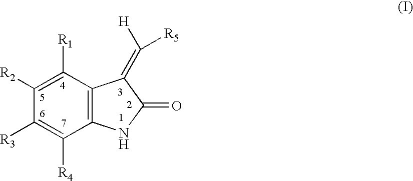

where

(a) R₁, R₂, R₃, and R₄ are selected from the group consisting of hydrogen, trihalomethyl, hydroxyl, amine, thioether, cyano, alkoxy, alkyl, amino, bromo, fluoro, chloro, iodo, mercapto, thio, cyanoamido, alkylthio, aryl, heteroaryl, carboxyl, ester, oxo, alkoxycarbonyl, alkenyl, alkoxy, nitro, alkoxyl, and amido moieties; and

(b) R₅ is an optionally substituted aryl or heteroaryl cyclic moiety;

or a pharmaceutically acceptable salt, ester, amide, prodrug, isomer, or metabolite thereof.

The term “pharmaceutically acceptable salt” refers to a formulation of a compound that does not abrogate the biological activity and properties of the compound. Pharmaceutical salts can be obtained by reacting a compound of the invention with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “ester” refers to a chemical moiety with formula —(R)n-COOR′, where R and R′ are independently selected from the group consisting of saturated or unsaturated alkyl and homocyclic or heterocyclic ring moieties and where n is 0 or 1.

The term “saturated alkyl” refers to an alkyl moiety that does not contain any alkene or alkyne moieties. The alkyl moiety may be branched or non-branched.

The term “unsaturated alkyl” refers to an alkyl moiety that contains at least one alkene or alkyne moiety. The alkyl moiety may be branched or non-branched.

The term “homocyclic” refers to a compound which contains one or more covalently closed ring structures, and that the atoms forming the backbone of the ring are all carbon atoms.

The term “heterocyclic” refers to a compound which contains one or more covalently closed ring structures, in which the ring backbone contains at least one atom which is different from carbon.

The term “amide” refers to a chemical substituent of formula —NHCOR, where R is selected from the group consisting of hydrogen, alkyl, hydroxyl, and homocyclic or heterocyclic ring moieties, where the ring is optionally substituted with one or more substituents independently selected from the group consisting of alkyl, halogen, trihalomethyl, carboxylate, nitro, or ester.

The term “prodrug” refers to an agent that is converted into the parent drug in vivo. Prodrugs may be easier to administer than the parent drug in some situations. For example, the prodrug may be bioavailable by oral administration but the parent is not, or the prodrug may improve solubility to allow for intravenous administration.

The term “isomer” refers to a compound that has the same molecular formula as a second compound but is structurally or chemically different than the second compound. Isomers contain the same numbers of the same kinds of atoms, but the atoms are attached to one another in different ways.

The term “metabolite” refers to a compound that is a product of metabolism.

The indolinone compounds of the invention are preferably selected from the group consisting of

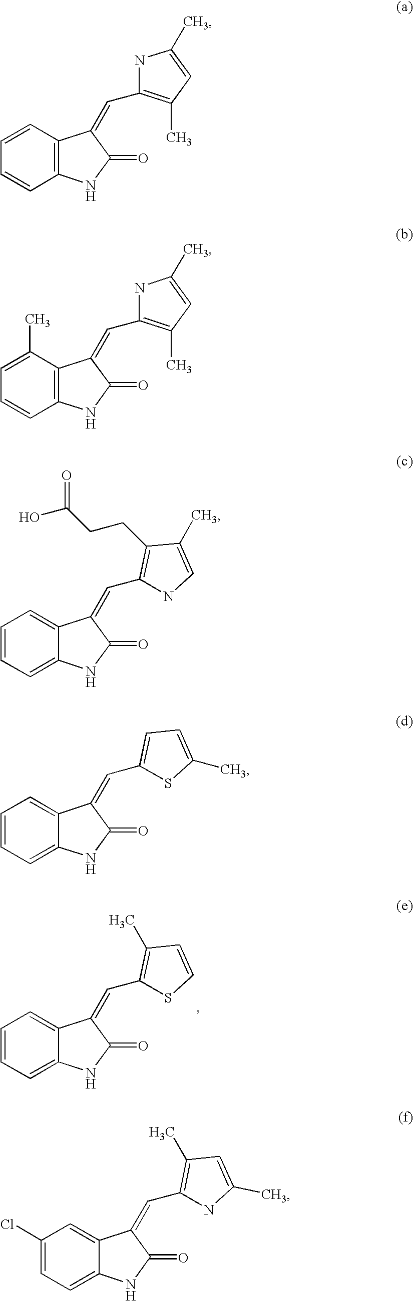

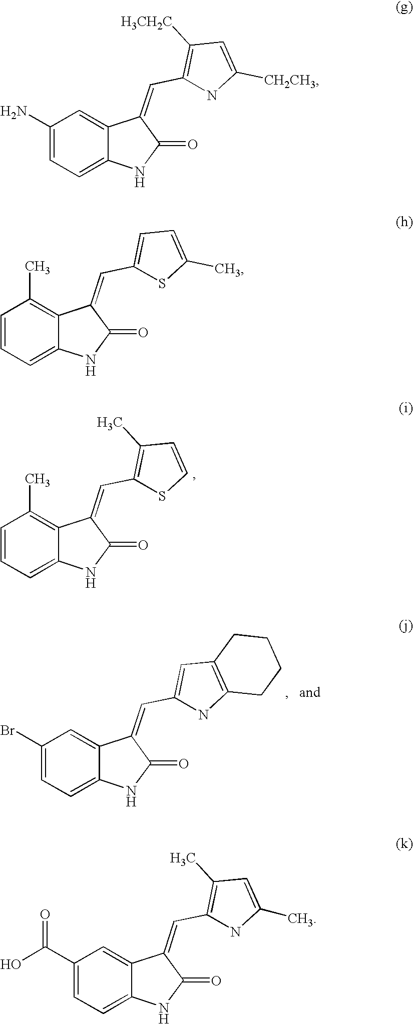

In a preferred embodiment, the invention provides a method for determining an efficacious dose of a drug administered to a subject for the purpose of modulating angiogenesis, where the monitoring occurs prior to, after, or during the administration of the drug. The monitoring may be continuous or non-continuous.

The marker of the invention is preferably present in a sample obtained from the subject. The sample is selected from the group consisting of whole blood, a blood fraction, blood plasma, blood serum, cells isolated from blood, whole urine, a urine fraction, saliva, cells isolated from saliva, spinal fluid, amniotic fluids, and biopsy of endothelial cells. The sample preferably comprises monocytes or endothelial cells.

The term “whole blood”, as used herein, refers to blood as obtained from the subject that has not undergone a further purification step. The term includes blood that is subsequently processed, such as by heating, freezing, or lysing, and the like, but excludes blood that is further purified.

The term “blood fraction”, as used herein, refers to blood as obtained from the subject that has undergone a further purification step. The term includes blood that is subsequently processed, such as by heating, freezing, or lysing, and the like.

The term “whole urine”, as used herein, refers to urine as obtained from the subject that has not undergone a further purification step. The term includes urine that is subsequently processed, such as by heating, freezing, or lysing, etc., but excludes urine that is further purified.

The term “urine fraction”, as used herein, refers to urine as obtained from the subject that has undergone a further purification step. The term includes urine that is subsequently processed, such as by heating, freezing, or lysing, etc.

In another preferred embodiment, the invention provides a method for determining an efficacious dose of a drug administered to a subject for the purpose of modulating angiogenesis, comprising the step of adding a receptor agonist to the sample. The receptor agonist preferably enhances angiogenesis, and it is preferably vascular endothelial cell growth factor.

In preferred embodiments, the invention provides a method for determining an efficacious dose of a drug administered to a subject for the purpose of modulating angiogenesis, where the marker is selected from the group consisting of cell division, cell motility, cell proliferation, cell death, cell survival, cell differentiation, protein phosphorylation, protein expression, protein glycosylation, mRNA expression, cellular membrane potential, DNA division, DNA methylation, and post-translational modification of a protein.

The term “cell motility” refers to the movement of a cell or cells and includes cell migration as a result of metastasis. Metastasis is the process by which a cancer cell invades tissues of the body. Cells can metastasize by separating from a primary tumor and traveling through the organism, for example, by traveling via the blood stream of the organism, and depositing in another region of the organism.

The term “protein phosphorylation”, as used herein, refers to an increase in phosphate content of a protein. This phosphorylation commonly occurs on tyrosine, serine, and/or threonine residues, but can occur on other amino acid residues, for example, histidine, as well.

The term “protein expression”, as used herein, refers to the production of a protein.

The term “protein glycosylation”, as used herein, refers to the addition of sugar or carbohydrate moieties to a protein.

The term “post-translational modification of a protein” refers to a change that occurs to a protein following the translation of the protein. Examples of post-translational modification include, but are not limited to, phosphorylation, acylation, and glycosylation.

The term “cellular membrane potential” refers to the relative concentration of ions between the inside and outside of a cell.

The term “DNA division” refers to the process that occurs when cells divide to ensure that the resulting cells have a complete copy of the original DNA.

The term “DNA methylation” refers to the incorporation of methyl groups into DNA.

The term “polynucleotide” refers to a multimer of nucleotides; specifically ribose sugar-based nucleotides, which include the following compounds: adenine, thymine, guanine, cytosine, or uracil. A polynucleotide may be a molecule of DNA or RNA.

The term “protein” refers to more than one amino acid joined together via a peptide bond. The phrase “peptide bond” represents the joining of two amino acids, whereby the carboxy terminus of one amino acid is joined to the amino terminus of another amino acid via an amide bond.

The invention further provides for a marker which is selected from the group consisting of DNA, RNA, mRNA, and protein. The marker can more preferably be selected, for example, from the group consisting of tissue factor, CD40, u-PA, ETS-1, IL8, and t-PA.

The term “tissue factor” refers to a lipoprotein that is released upon trauma to blood vessels. Tissue factor serves as a stimulatory protein in the extrinsic pathway for blood coagulation. Tissue factor and its clinical ramifications are well-known in the art. See, for example, Zhang, Y. et al. J. Clin. Invest. 94:1320, 1994, and reference cited therein.

In a preferred embodiment, the step of monitoring a marker comprises the step of determining the presence or the amount of the marker or the activity of the marker. The presence or the amount of the marker is preferably detected using an antibody. The activity of the marker is preferably determined by measuring blood clotting.

The term “antibody” refers to polypeptides which contain regions that can bind other polypeptides. Antibodies can be either polyclonal or monoclonal, or fragments thereof.

The term “polyclonal” refers to antibodies that are heterogenous populations of antibody molecules derived from the sera of animals immunized with an antigen or an antigenic functional derivative thereof. For the production of polyclonal antibodies, various host animals may be immunized by injection with the antigen. Various adjuvants may be used to increase the immunological response, depending on the host species.

“Monoclonal antibodies” are substantially homogenous populations of antibodies to a particular antigen. They may be obtained by any technique which provides for the production of antibody molecules by continuous cell lines in culture. Monoclonal antibodies may be obtained by methods known to those skilled in the art. See, for example, Kohler, et al., Nature 256:495-497,1975, and U.S. Pat. No. 4,376,110.

In another preferred embodiment, the step of monitoring a marker comprises the step of determining the presence or amount of marker mRNA. The presence or amount of marker mRNA is preferably determined using reverse transcriptase polymerase chain reaction or a polynucleotide probe.

The marker is preferably selected from the group consisting of tissue factor, CD40, u-PA, ETS-1, IL8, and t-PA and is preferably present in a sample obtained from a subject.

The phrase “reverse transcriptase polymerase chain reaction procedures”, as used herein, refers to procedures known to one in the art for amplifying low level amounts of polynucleotides.

The term “reverse transcriptase”, as used herein, refers to an enzyme capable of creating a DNA template of polynucleotides corresponding to any fragment of a messenger RNA template of polynucleotides of any species. Those in the art commonly refer to the DNA template described above as a “cDNA”, which stands for complementary DNA.

In a preferred embodiment, the step of monitoring a marker comprises the step of comparing the marker to a standard.

The term “standard”, as used herein, refers to a known element against which an unknown element is compared and judged. This may include a single known element against which the unknown element is compared and judged. Conversely, there may be a range of known elements against which the unknown element is judged. This range of elements may be considered a standard curve.

The term “element” as used herein refers to a physical characteristic that is determinable as well as a numerical or graphical value that represents what is actually determined.

In another aspect, the invention provides an efficacious dose of a drug, where the efficacious dose is determined using any of the methods described in this application. This efficacious dose of a drug is preferably to be used in a method of treating a disease, most preferably a disease related to angiogenesis.

The summary of the invention described above is non-limiting and other features and advantages of the invention will be apparent from the following description of the preferred embodiments, and from the claims.

### DETAILED DESCRIPTION OF THE INVENTION

The invention is directed in part towards determining an efficacious dose of protein kinase inhibitors that modulate the rate of growth of tumors by severing their sources of sustenance. The inhibitors are designed to specifically bind protein kinases expressed in the vasculature that supply tumors with sustenance. One such protein kinase target is FLK-1, which is over-expressed in the proliferating endothelial cells of a growing tumor, but not in the surrounding quiescent endothelial cells. Plate et al., 1992, Nature 359:845-848.

Recently, the focus on developing new drugs has been on compounds that interact with biological molecules involved in the regulation of the aberrant processes. Since the mechanism of these drugs is not based on cell death, but on cell regulation, it would be inappropriate to determine an effective dose of these drugs guided by toxicity. Thus, there is a need in the art for a novel method for determining the efficacious dose of a drug based on monitoring the activity that is to be regulated.

However, it is often difficult to directly monitor the aberrant process of interest. Consequently, researchers and clinicians have begun to monitor surrogate markers of the process of interest. By monitoring these surrogate markers it is possible to monitor the underlying process of interest.

For example, surrogate markers have proven useful in the diagnosis and staging of various forms of cancer (see, for example, Adamson, A. S., et al., British Journal of Urology 71:587, 1993). In some instances surrogate markers have been useful in monitoring the progress of cancer treatment, but the drugs used work directly on tumor cells (see, for example, Zhang, Y., et al., J. Clin. Invest. 94:1320, 1994).

Consequently, there remains a need for a novel method of determining the efficacious dose of a drug based upon monitoring the activity that is to be regulated. Specifically this long felt need can be a novel method for determining the efficacious dose of a drug based on monitoring a surrogate marker of the activity that is to be regulated, since in some cases the direct effect on the tumors cannot be seen and conventional markers cannot be used.

FLK-1 protein kinase is activated upon binding VEGF, a strong regulator for endothelial cell proliferation as well as normal and pathological angiogenesis. Klagsbum and Soker, Current Biology 3:699-702, 1993. Thus, compounds that specifically inhibit the FLK-1 protein kinase are potential anti-cancer agents as they may decrease the vasculature that nourishes tumors and arrest the growth of tumors. In addition, compounds that specifically inhibit FLK-1 will potentially represent a new generation of cancer therapeutics as they will most likely cause few side effects. These potential properties are a welcome improvement over the currently utilized cancer therapeutics that cause multiple side effects and deleteriously weaken patients.

I. Target Diseases to be Treated by Methods of the Invention

Blood vessel proliferative disorders refer to angiogenic and vasculogenic disorders generally resulting in abnormal proliferation of blood vessels. The formation and spreading of blood vessels play important roles in a variety of physiological processes such as embryonic development, corpus luteum formation, wound healing and organ regeneration. They also play a pivotal role in continued growth of tumor mass and cancer development. Other examples of blood vessel proliferation disorders include arthritis, where new capillary blood vessels invade the joint and destroy cartilage, and ocular diseases, like diabetic retinopathy, where leaky new capillaries in the retina invade the vitreous and bleed, causing an inflammatory response and detachment of the retina and, subsequently, cause blindness. Conversely, disorders related to the shrinkage, contraction or closing of blood vessels are implicated in such diseases as restenosis.

Fibrotic disorders refer to the abnormal formation of extracellular matrix. Examples of fibrotic disorders include hepatic cirrhosis and mesangial cell proliferative disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis.

Mesangial cell proliferative disorders refer to disorders brought about by abnormal proliferation of mesangial cells. Mesangial proliferative disorders include various human renal diseases, such as glomerulonephritis, diabetic nephropathy, malignant nephrosclerosis, thrombotic microangiopathy syndromes, transplant rejection, and glomerulopathies. PDGF-R has been implicated in the maintenance of mesangial cell proliferation. Floege et al., Kidney International 43:47S-54S, 1993.

Protein tyrosine kinases (PTKs) are directly associated with the cell proliferative disorders described above. For example, some members of the receptor PTK family have been associated with the development of cancer. Some of these receptors, like EGFR (Tuzi et al., Br. J. Cancer 63:227-233, 1991; Torp et al., APMIS 100:713-719, 1992) HER2/neu (Slamon et al., Science 244:707-712, 1989) and PDGF-R (Kumabe et al., Oncogene 7:627-633, 1992) are over-expressed in many tumors and/or persistently activated by autocrine loops. In fact, PTK over-expression (Akbasak and Suner-Akbasak et al., J. Neurol. Sci. 111:119-133, 1992; Dickson et al., Cancer Treatment Res. 61:249-273, 1992; Korc et al., J. Clin. Invest. 90:1352-1360, 1992) and autocrine loop stimulation (Lee and Donoghue, J. Cell. Biol. 118:1057-1070, 1992; Korc et al., supra; Akbasak and Suner-Akbasak et al., supra) account for the most common and severe cancers. For example, EGFR is associated with squamous cell carcinoma, astrocytoma, glioblastoma, head and neck cancer, lung cancer and bladder cancer. HER2 is associated with breast, ovarian, gastric, lung, pancreas and bladder cancer. PDGF-R is associated with glioblastoma, lung, ovarian, melanoma and prostate cancer. The receptor PTK c-met is generally associated with hepatocarcinogenesis and thus hepatocellular carcinoma. Additionally, c-met is linked to malignant tumor formation. More specifically, c-met has been associated with, among other cancers, colorectal, thyroid, pancreatic and gastric carcinoma, leukemia and lymphoma. Additionally, over-expression of the c-met gene has been detected in patients with Hodgkins disease, Burkitts disease, and the lymphoma cell line.

The IGF-I receptor PTK, in addition to being implicated in nutritional support and in type-II diabetes, is also associated with several types of cancers. For example, IGF-I has been implicated as an autocrine growth stimulator for several tumor types, e.g. human breast cancer carcinoma cells (Arteaga et al., J. Clin. Invest. 84:1418-1423, 1989) and small cell lung tumor cells (Macauley et al., Cancer Res. 50:2511-2517, 1990). In addition, IGF-I, integrally involved in the normal growth and differentiation of the nervous system, appears to be an autocrine stimulator of human gliomas. Sandberg-Nordqvist et al., Cancer Res. 53:2475-2478, 1993. The importance of the IGF-IR and its modulators in cell proliferation is further supported by the fact that many cell types in culture (fibroblasts, epithelial cells, smooth muscle cells, T-lymphocytes, myeloid cells, chondrocytes, osteoblasts, the stem cells of the bone marrow) are stimulated to grow by IGF-I. Goldring and Goldring, Eukaryotic Gene Expression 1:301-326, 1991. A series of recent publications suggest that IGF-IR plays a central role in the mechanisms of transformation and, as such, could be a preferred target for therapeutic interventions for a broad spectrum of human malignancies. Baserga, Cancer Res. 55:249-252, 1995; Baserga, Cell 79:927-930, 1994; Coppola et al., Mol. Cell. Biol. 14:4588-4595, 1994.

The association between abnormalities in receptor PTKs and disease are not restricted to cancer, however. For example, receptor PTKs are associated with metabolic diseases like psoriasis, diabetes mellitus, wound healing, inflammation, and neurodegenerative diseases. EGF-R is implicated in corneal and dermal wound healing. Defects in Insulin-R and IGF-IR are implicated in type-II diabetes mellitus. A more complete correlation between specific receptor PTKs and their therapeutic indications is set forth in Plowman et al., DN&P 7:334-339, 1994.

Non-receptor PTKs, including src, abl, fps, yes, fyn, lyn, lck, blk, hck, fgr, yrk (reviewed by Bolen et al., FASEB J. 6:3403-3409, 1992), are involved in the proliferative and metabolic signal transduction pathways also associated with receptor PTKs. Therefore, the present invention is also directed towards designing modulators against these classes of PTKs. For example, mutated src (v-src) is an oncoprotein (pp60^{v-src}) in chicken. Moreover, its cellular homolog, the proto-oncogene pp60^{c-src} transmits oncogenic signals of many receptors. For example, over-expression of EGF-R or HER2/neu in tumors leads to the constitutive activation of pp60^{c-src}, which is characteristic of the malignant cell but absent in the normal cell. On the other hand, mice deficient for the expression of c-src exhibit an osteoporotic phenotype, indicating a key participation of c-src in osteoclast function and a possible involvement in related disorders. Similarly, Zap 70 is implicated in T-cell signaling. Both receptor PTKs and non-receptor PTKs are connected to hyper-immune disorders.

The instant invention is directed in part towards designing modulators of PTK function that could indirectly arrest the growth of tumors by cutting off their source of sustenance. Normal vasculogenesis and angiogenesis play important roles in a variety of physiological processes such as embryonic development, wound healing, organ regeneration and female reproductive processes such as follicle development in the corpus luteum during ovulation and placental growth during pregnancy. Folkman and Shing, J. Biological Chem. 267:10931-34, 1992. However, many diseases are driven by persistent unregulated or inappropriate angiogenesis. For example, in arthritis, new capillary blood vessels invade the joint and destroy the cartilage. In diabetes, new capillaries in the retina invade the vitreous, bleed and cause blindness. Folkman, 1987, in: Congress of Thrombosis and Haemostasis (Verstraete, et. al, eds.), Leuven University Press, Leuven, pp.583-596. Ocular neovascularization is the most common cause of blindness and occurs in approximately twenty (20) eye diseases.

In addition, angiogenesis is an important factor in the pathophysiology of endometriosis, a condition characterized by implantation of ectopic endometrium in the peritoneal cavity. Elevated levels of VEGF are found in peritoneal fluid of patients with endometriosis. During the menstrual cycle, expression of Flt is constant but that of kinase domain receptor (KDR) is increased in the luteal phase, at which time the cells migrate in response to VEGF. KDR expression and the migratory response are significantly higher in patients with endometriosis (McLaren, J., et al., J. Clin. Invest. 98:482, 1996).

Moreover, vasculogenesis and/or angiogenesis can be associated with the growth of malignant solid tumors and metastasis. A tumor must continuously stimulate the growth of new capillary blood vessels for the tumor itself to grow. Furthermore, the new blood vessels embedded in a tumor provide a gateway for tumor cells to enter the circulation and to metastasize to distant sites in the body. Folkman, J. Natl. Cancer Inst. 82:4-6, 1990; Klagsbrunn and Soker, Current Biology 3:699-702, 1993; Folkman, J. Natl., Cancer Inst. 82:4-6, 1991; Weidner, et al., New Engl. J. Med. 324:1-5, 1991.

Several polypeptides with in vitro endothelial cell growth promoting activity have been identified. Examples include acidic and basic fibroblast growth factor (aFGF, bFGF), vascular endothelial growth factor (VEGF) and placental growth factor. Unlike aFGF and bFGF, VEGF has recently been reported to be an endothelial cell specific mitogen. Ferrara and Henzel, Biochem. Biophys. Res. Comm. 161:851-858, 1989; Vaisman et al., J. Biol. Chem. 265:19461-19566, 1990.

Thus, identifying the specific receptors that bind FGF or VEGF is important for understanding endothelial cell proliferation regulation. Two structurally related receptor PTKs that bind VEGF with high affinity are identified: the FLT-1 receptor (Shibuya et al., Oncogene 5:519-524, 1990; De Vries et al., Science 255:989-991, 1992) and the KDR/FLK-1 receptor, discussed in the U.S. patent application Ser. No. 08/193,829. In addition, a receptor that binds aFGF and bFGF is identified. Jaye et al., Biochem. Biophys. Acta 1135:185-199, 1992). Consequently, these receptor PTKs most likely regulate endothelial cell proliferation.

FGFRs play important roles in angiogenesis, wound healing, embryonic development, and malignant transformation. Basilico and Moscatelli, Adv. Cancer Res. 59:115-165, 1992. Four mammalian FGFRs (FGFR1-4) have been described and additional diversity is generated by alternative RNA splicing within the extracellular domains. Jaye et al., Biochem. Biophys. Acta 1135:185-199, 1992. Like other receptor PTKs, dimerization of FGF receptors is essential for their activation. Soluble or cell surface-bound heparin sulfate proteoglycans act in concert with FGF to induce dimerization (Schlessinger et al., Cell 83:357-360, 1995), which leads to autophosphorylation of specific tyrosine residues in the cytoplasmic domain. Mohammadi et al., Mol. Cell Biol. 16:977-989, 1996.

Mutations in three human FGF receptor genes, FGFR1, FGFR2, and FGFR3, have been implicated in a variety of human genetic skeletal disorders. Mutations in FGFR1 and FGFR2 result in the premature fusion of the flat bones of the skull and cause the craniosynostosis syndromes, such as Apert (FGFR2) (Wilkie et al., Nat. Genet. 8:269-274, 1994), Pfeiffer (FGFR1 and FGFR2) (Muenke et al., Nat. Genet. 8:269-274, 1994), Jackson-Weiss (FGFR2) (Jabs et al., Nat. Genet. 8:275-279, 1994) and Crouzon (FGFR2) (Jabs et al., Nat. Genet. 8:275-279, 1994) syndromes. In contrast mutations in FGFR3 are implicated in long bone disorders and cause several clinically related forms of dwarfism including achondroplasia (Shiang et al., Cell 78:335-342, 1994), hypochondroplasia (Bellus et al., Nat. Genet. 10:357-359, 1995) and the neonatal lethal thanatophoric dysplasia (Tavormina et al., Nat. Genet. 9:321-328, 1995). It has been shown that these mutations lead to constitutive activation of the tyrosine kinase activity of FGFR3 (Webster et al., EMBO J. 15:520-527, 1996). Furthermore gene-targeting experiments in mice have revealed an essential role for FGFR3 in developmental bone formation (Deng et al., Cell 84:911-921, 1996).

Another major role proposed for FGFs in vivo is the induction of angiogenesis (Folkman and Klagsbrunn, Science 236:442, 1987). Therefore, inappropriate expression of FGFs or of their receptors or aberrant function of the tyrosine kinase activity or their receptors could contribute to several human angiogenic pathologies such as diabetic retinopathy, rheumatoid arthritis, atherosclerosis and tumor neovascularization (Klagsbrunn and Edelman, Arteriosclerosis 9:269, 1989). Moreover, FGFs are thought to be involved in malignant transformation. Indeed, the genes coding for the three FGF homologues int-2, FGF-5 and hst-1/K-fgf were originally isolated as oncogenes. Furthermore, the cDNA encoding FGFR1 and FGFR2 are amplified in a population of breast cancers (Adnane et al., Oncogene 6:659-663, 1991). Over-expression of FGF receptors has been also detected in human pancreatic cancers, astrocytomas, salivary gland adenosarcomas, Kaposi sarcomas, ovarian cancers and prostate cancers.

Evidence, such as the disclosure set forth in copending U.S. application Ser. No. 08/193,829, incorporated herein by reference, including all the drawings, strongly suggests that VEGF is not only responsible for endothelial cell proliferation, but also is a prime regulator of normal and pathological angiogenesis. See generally, Klagsbrunn and Soker, Current Biology 3:699-702, 1993; Houck et al., J. Biol. Chem. 267:26031-26037, 1992. Moreover, it has been shown that KDR/FLK-1 and flt-1 are abundantly expressed in the proliferating endothelial cells of a growing tumor, but not in the surrounding quiescent endothelial cells. Plate et al., Nature 359:845-848, 1992; Shweiki et al., Nature 359:843-845, 1992.

II. Pharmaceutical Compositions and Administration of Drugs

Methods of preparing pharmaceutical formulations of the compounds, methods of determining the amounts of compounds to be administered to a patient, and modes of administering compounds to an organism are disclosed in International Patent Publication No. WO 96/22976, published Aug. 1, 1996 by Ballinari et al., which is incorporated herein by reference in its entirety, including any drawings. Those skilled in the art will appreciate that such descriptions are applicable to the present invention and can be easily adapted to it. The mechanism of such action and possible uses for such compounds are described in International Patent Publication WO 96/40116, published Dec. 19, 1996 by Tang et al., and entitled “Indolinone Compounds for the Treatment of Disease” (Lyon & Lyon Docket No. 223/298), incorporated herein by reference, including any drawings.

The compounds described herein can be administered to a human patient per se, or in pharmaceutical compositions where they are mixed with suitable carriers or excipient(s). Techniques for formulation and administration of the compounds of the instant application may be found in “Remington's Pharmaceutical Sciences,” Mack Publishing Co., Easton, Pa., latest edition or in International Patent Publication No. WO 96/40116, published Dec. 19, 1996 by Tang et al., and entitled “Indolinone Compounds for the Treatment of Disease” (Lyon & Lyon Docket No. 223/298)

A. Effective Dosage

Pharmaceutical compositions suitable for use in the present invention may include compositions wherein the active ingredients can be contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein and in International Patent Publication No. WO 96/40116, published Dec. 19, 1996 by Tang et al., and entitled “Indolinone Compounds for the Treatment of Disease” (Lyon & Lyon Docket No. 223/298)

B. Packaging

The compositions may, if desired, be presented in a pack or dispenser device which may contain one or more unit dosage forms containing the active ingredient according to the description provided in International Patent Publication No. WO 96/40116, published Dec. 19, 1996 by Tang et al., and entitled “Indolinone Compounds for the Treatment of Disease” (Lyon & Lyon Docket No. 223/298).

III. Vascular Endothelial Growth Factor (VEGF)

An important regulatory molecule in the process of angiogenesis is vascular endothelial growth factor (VEGF). VEGF is a hormone that induces angiogenesis. Immunohistochemical results indicate that significant levels of VEGF are present in prostate cancer, but not in benign prostatic hyperplasia (BPH) or normal prostate cells in vivo (Ferrer, F. A., et al. J. Urol. 157:2329, 1997). The prominence of VEGF mRNA levels in human ovarian and endometrial tumors demonstrates that VEGF may be involved in promoting tumor angiogenesis and stroma generation, acting as an endothelial cell mitogen (Doldi, N., et al. Gynecol. Endocrinol. 10:375, 1996). VEGF is also thought to contribute to neovascularization of human renal cell carcinomas (RCCs) through a paracrine mechanism, and could be used as a relevant indicator of angiogenesis (Sato, K., et al. Gan Ko Kagaku Ryoho 24:389, 1997). Two receptor tyrosine kinases, Flt-1 and Flk-1 have been identified that induce angiogenesis in response to VEGF stimulation.

In addition to inducing angiogenesis, VEGF induces the expression of tissue factor on endothelial cells and monocytes through the activation of these receptors. (Flk-1 and Flt-1 are present on endothelial cells whereas only Flt-1 is present on monocytes). Consequently, tissue factor production by monocytes may be used as a surrogate marker for monitoring activity of angiogenesis inhibitors on endothelial cell function related to Flk-1 or Flt-1.

By monitoring a surrogate marker of the process of interest, it may be possible to monitor the process of interest. Thus, it may be possible to determine the efficacious dose of a drug that acts upon the process of interest, by monitoring the rate of change of surrogate marker production following drug treatment.

There are many advantages of the method of the present invention. For instance, it may allow one to monitor an otherwise unmonitorable process. Also, where the drug is not intended to be cytotoxic, it may allow a better dose determination than current methods since one may be able to detect efficacy by means other than monitoring for effects of toxicity, thereby possibly avoiding or minimizing negative side effects for the patient. Since the dosage of the drug is not measured by cytotoxicity by the methods of the invention, a lower dose may be established which does not cause significant toxic side effects. These are just a few examples of the advantages of the method of the present invention.

IV. Other Markers

A number of other markers are shown to be effective for the purpose of practicing the present invention. Immunohistological studies of endothelial cells show a widespread, even distribution of the immunoregulative molecule CD40 in tumor capillaries and suggest that within renal cell carcinoma, the appearance of endothelial CD40 may be related to angiogenesis. Kluth, B., et al. Cancer Res. 57:891, 1997.

The binding of urokinase-type plasminogen activator u-PA to a specific cell surface receptor (u-PA-R) has been shown to enhance plasminogen activation, a process involved in extracellular matrix degradation and cell migration during angiogenesis and tumor growth. Studies show an active invasive phenotype of endothelial cells in renal cell carcinoma and suggest a role for the plasminogen activation system in tumoral angiogenesis and invasion. Xu, Y., et al. Hum. Pathol. 28:206, 1997. VEGF stimulates uPA activity in vascular endothelial cells. Mandriota, et al., J. Biol. Chem. 270:9709, 1995.

The transcription factor ETS-1 is a member of the ets gene family of transcription factors, which binds to the Ets binding motif in the cis-acting elements and regulates the expression of certain genes. ETS-1 plays an important role in angiogenesis, regulating the expression of proteases and the migration of endothelial cells. Typical angiogenic growth factors, such as VEGF, induce the expression of ETS-1 mRNA in human umbilical vein endothelial cells (HUVECs), immortalized HUVECs, or human omental microvascular endothelial cells (HOMECs). Iwasaka, et al., J. Cell. Physiol. 169:522, 1996.

Tissue plasminogen activator t-PA converts plasminogen to plasmin during activation of the fibrinolytic system. t-PA is predominantly released from endothelial cells. In some cancers, such as acute nonlymphocytic leukemias, poor outcome correlates with high t-PA levels. Bell, W. R., Semin. Thromb. Hemost. 22:459, 1996. The release of t-PA is induced by VEGF. Carmeliet, P., et al., Ann. N.Y. Acad. Sci. 811:191, 1997.

The foregoing enumerated a list of some of the possible markers that can be used with the techniques of the present invention. Those skilled in the art realize that this list is non-exhaustive and other markers can be used to practice the invention. The references listed in this section are hereby incorporated herein by reference, including any drawings.

V. Monitoring the Markers

A number of techniques to monitor the markers are known in the art. Spectrophotometrical determination after the addition of a specific chromogenic substrate (see, for example, Adamson, A. S., et al., British Journal of Urology 71:587, 1993); detection with antibodies (see, for example, Clauss, M., et al., FEBS Letters 390:224, 1996); two stage clotting assay (see, for example, Hu, T., et al., Thrombosis Research 72:155, 1993); one-stage recalcification assay (see, for example, Falanga, A., et al., Blood 86:1072, 1995); enzyme-linked immunosorbent assay (ELISA) (see, for example, Luther, T., et al., Nature Medicine 2:491, 1996); solid-phase enzyme immunoassay employing polyclonal antisera (CEA-EIA) (see, for example, Fritsche, H. A., Clin. Chem 39:2431, 1993); hydrogen peroxide assay (see, for example, Conkling, P. R., et al., Cancer Research 48:5604, 1988); and measurement of tissue factor mRNA levels in endothelial cells (Potgens, A., et al., Thrombosis and Hemostasis 72:208, 1994) are examples of the types of monitoring methods available to one skilled in the art. The references listed in this section are hereby incorporated herein by reference, including any drawings.

VI. Generation of a Standard Curve to Determine the Efficacious Dose of a Drug

A standard curve can be generated by monitoring in vitro or in vivo samples. The standard curve provides the relationship between the amount of the marker released per a known number of cells as a function of the dose of a drug. As the dose of the drug increases, the amount of marker may decrease up to the point where a saturating level of efficacy has been achieved. At this point, the curve may flatten out, indicating that additional amounts of the drug have little impact (i.e. a change of less than 5% or within the standard error of acceptable assay detection) on the therapeutic effect. The standard curve can show with accuracy where this point is reached. Thus, the efficacious dose may relate to three different points on the standard curve: the point where the curve begins to slope downward, i.e. the minimal efficacious dose, the point where the curve flattens out, i.e. the maximal efficacious dose, and the point between the minimal and the maximal dose, i.e. the IC₅₀ value.

In addition, parallel aliquots of samples may be re-stimulated in vitro with VEGF with and without the drug in order to measure the sensitivity of the blood sample toward further VEGF stimulation. When no further changes in marker level in the parallel in vitro challenge can be detected, then a maximal level of efficacy has been achieved.

In vitro samples can be prepared from solutions comprising endothelial cells or monocytes and then monitoring the amount of the marker with respect to the dose of a drug. The in vitro samples can be prepared in simulated blood conditions, the creation of which are well known to those skilled in the art. It can be determined whether the amount of marker released in these conditions corresponds to the amount of marker released in a patient who has been administered a drug. In vivo samples can be prepared from blood samples taken from a patient who has been administered a drug of a known dosage.

The slope of the standard curve and the point where the maximal efficacy has been reached may be dependent upon the storage conditions of the blood sample before it is assayed by the methods of the invention. For instance, a blood sample stored in a solution comprising citrate may yield a different slope or efficacious point than a blood sample stored in a solution comprising ethylenediaminetetraacetic acid (EDTA). Determination of the maximal efficacy of a drug is accurate when based on a standard curve described herein when the storage conditions of the blood sample of the patient are the same as the storage conditions of the blood samples of patients used to construct the standard curves. Alternatively, tests can be done to prepare normalization factors so that samples which were stored in a particular agent can be validly compared to a standard curve prepared by use of samples stored with another agent or a curve prepared by in vivo samples.

Thus, the efficacious doses of a drug are determined from the standard curve. Since the standard curve is a relation between the amount of the marker to the dose of the administered drug, the efficacious dose of a drug can be determined from the standard curve once the ratio of the amount of the marker to the dose of the administered drug is determined for the blood sample from the patient of interest.

VII. Circulating Levels of Tissue Factor

A. Angiogenesis

It may be possible to monitor angiogenesis by monitoring circulating levels of tissue factor. Thus, it may be possible to determine the effective dose of a drug on angiogenesis by monitoring tissue factor.

In one manner, the total plasma levels of tissue factor measured could be determined before and after treatment with an anti-angiogenic drug. The difference in the levels of tissue factor in plasma of subjects before and after treatment with an anti-angiogenic drug would reflect the effect of this drug on angiogenesis.

B. Tumors

When a subject bears tumors that produce tissue factor it may be possible to monitor the level of tissue factor produced by these tumors. Tissue factor can become a marker for tumor growth. Thus, one potentially can monitor tumor progression, drug efficacy, and potentially staging and/or decreases in tumor burden, using this marker.

It may also be possible to determine the effective dose of a drug for treatment of these tumors by monitoring tissue factor.

In one manner, an efficacious dose of an anti-tumorigenic drug can be determined. When appropriate, total plasma levels of tissue factor measured may be predominantly or even exclusively produced by tumor cells. Therefore, the differences in levels of tissue factor in plasma of subjects before and after treatment with an anti-tumorigenic drug may reflect the activity of this drug on tumor growth.

In another manner, an efficacious dose of an anti-angiogenic drug can be determined. When total plasma levels of tissue factor measured are predominantly or even exclusively produced by tumor cells, then the differences in levels of tissue factor in plasma of subjects before and after treatment with an anti-angiogenic drug may reflect the activity of this drug on tumor growth via the inhibition of angiogenesis and not as a direct effect on tumor cell growth.

In all manners above, tissue factor protein expression, presence and levels, can be determined in a variety of ways, a few of which are defined in the examples section below.

VIII. Expression of Tissue Factor mRNA or Protein by Monocytes

The differences in production of tissue factor mRNA or protein by monocytes from patients before and after treatment with the drug may reflect the exposure of tissues and cells of the patients to biologically active concentrations of this drug. As such, it may be possible to determine an efficacious dose of a drug by monitoring the differences in production of tissue factor mRNA or protein by monocytes in subjects before and after treatment with the drug.

Attempts to quantify the biologically active levels of a drug may be done by first establishing a standard curve of the inhibitory activity of this drug (expressed as % inhibition) on VEGF-induced production of tissue factor protein or mRNA by monocytes from patients having normal angiogenic function in vitro. The VEGF levels used for these studies may be saturating levels. The length of incubation of monocytes with VEGF and the drug of interest may be one that is reflective of the clinical dosage regimen. Using the % inhibition of tissue factor production observed in patient monocytes before and after treatment, the theoretical concentrations of biologically active drug that the patient's monocytes have been exposed to in situ can be extrapolated (such as that used in a standard RIA kit).

This represents the biological concentration of the drug on target. This level can be compared to the actual levels of the drug measured in the blood (by HPLC analysis) of the treated subjects to potentially yield information on the relative levels of the drug circulating to that which is active on target.

Ideally, if monocytes can be isolated directly from a subject's blood in a rapid and high yield procedure, a pure population of cells is preferred for these studies. However, in consideration of the washout effect, the extensive isolation procedure would have on biological activity of the drug, peripheral blood lymphocytes/leukocytes (PBLs) could be used in place of purified monocytes in the interest of time.

It is suggested in the literature that monocytes are the primary source of tissue factor amongst the leukocyte subpopulations. Since only limited amounts of blood can be drawn from a subject in sequential time points, the levels of tissue factor protein produced may be low (yield can be 10⁵ monocytes/ml of whole blood). Hence, PCR detection of mRNA for tissue factor may be used instead of determining the expression of tissue factor protein.

In all aspects above, tissue factor protein expression (presence) or levels and mRNA presence or levels can be determined in a variety of ways, a few of which are defined below.

IX. Biological Activity of Indolinone Compounds

Indolinone compounds of the invention can be tested for their ability to activate or inhibit protein kinases in biological assays. The methods used to measure indolinone induced modulation of protein kinase function are described herein. Indolinone compounds of the invention were tested for their ability to inhibit the FLK-1 protein kinase. The biological assay and results of these inhibition studies are reported herein.

### EXAMPLES

The examples below are not limiting and are merely representative of various aspects and features of the present invention. The examples demonstrate methods of practicing the invention.

### Example 1

Collection of Blood Samples

Blood samples for serum chemistry, hematology and tissue factor quantification can be obtained from subjects enrolled in clinical studies prior to the first injection and at pre-determined intervals (determined by the turnover of the marker as well as pharmacokinetic properties of the drug in use) following the first injection.

Blood samples can be obtained prior to and following every cycle of treatment with the drug. Patients will receive drug treatment daily or at predetermined intervals within a week or weekly via, for example, intravenous, oral, or subcutaneous injections. Blood samples can be drawn, for example, from the contralateral arm or distant from a venous access device used for intravenous administration of the drug when applicable and possible. A blood sample for determination of the plasma level of the administered drug will also be drawn. Tumor assessment will occur prior to and following treatment with the drug.

### Example 2

Tissue Factor Sample Processing

1. Collect 3.0 mL of whole blood into sodium citrate treated tube at the designated times. Following collection gently invert the tube (15 times). Keep blood at room temperature.

2. Dilute whole blood 1:3 with normal saline. Layer the diluted blood gently into a separate tube containing a Ficoll-Hypaque gradient. (Use a Ficoll sodium metrizoate gradient with a density of 1.077 g/mL). Centrifuge at 400 g at 22-25° C. for 30 minutes.

3. Following centrifugation, collect diluted plasma into a clean tube and freeze. At a later time, quantify the level of tissue factor present in the sample by using an ELISA specific for human tissue factor (American Diagnostica Inc.; Greenwich, Conn.). The plasma level of the compound of interest can also be quantified using HPLC analysis.

4. Collect cells from the interface by means of a Pasteur pipette. Transfer to clean glass tube. Add normal saline to wash cells and remove Ficoll-Hypaque.

5. Centrifuge cells at 1000 g at 22-25° C. for 6 minutes. Aspirate off supernatant and discard. Freeze cell pellet with liquid nitrogen or in dry ice.

6. Thaw cell pellet and aliquot into two fractions for measurement of tissue factor mRNA and protein. Label the tubes with patient initials, ID number, collection date and time.

9. Store tubes at −70° C.

10. For detection of tissue factor mRNA, RNA is first isolated from resuspended cell pellets (Strategene RNA isolation kit). cDNA is then prepared from the RNA by reverse transcription. mRNA for tissue factor is then detected by PCR using primers specific for human tissue factor. The quantitative PCR assay is performed using a macrophage/monocyte marker, such as CD14, as a standard.

11. For measurement of expression of tissue factor protein on lysed PBLS, lysates from resuspended cell pellets are incubated with citrated human plasma and CaCl₂ and the appearance of the first clotting is noted. The levels of tissue factor expression on the lysed cells are then quantified using a standard curve constructed using purified tissue factor.

### Example 3

Assay of Purified Marker

This procedure is complementary to Example 2. The purified markers may include, but are not limited to, tissue factor, IL8, urokinase, and tPA. Thawed plasma samples, lysates from resuspended cell pellets, or cultured supernatants can be used. One can assay for the presence of the purified marker protein in the plasma or on the cell lysates by using specific ELISAs. Alternatively, one can assay for the presence of the mRNA for the marker protein by quantitative PCR assay, using a macrophage/monocyte marker, such as CD14, as a standard. An example of an ELISA assay for the purified marker (e.g. tissue factor) is illustrated in the following.

Procedure:

1. Add 100 μL. of sample (e.g. human plasma, endothelial cell, tumor cell, or monocyte cultured supernatants, freeze-thawed or detergent lysates from endothelial cells, monocytes, or PBL cells) or standard (0, 50, 100, 500, and 1000 pg/mL tissue factor) to a 96-well plate, and let sit for 3 hr.

2. Wash wells 4 times in wash buffer (PBS+0.1% triton X-100).

3. Add 100 μL biotinylated anti-human tissue factor F(ab′)₂ fragments and let sit for 1 hr.

4. Wash wells 4 times in wash buffer.

5. Add 100 μL streptavidin-horseradish peroxidase and let sit for 1 hr.

6. Wash wells 4 times in wash buffer.

7. Add 100 μL TMB substrate 15-30′.

8. Add 50 μL 0.5 M H₂SO₄ and read at 410 nm on an ELISA plate reader.

9. Construct a standard curve, and using 4 parameter curve-fit analysis calculate the amount of tissue factor present in the test samples.

### Example 4

Standard Curve

This procedure is complementary to Examples 2 and 3. The markers of interest may include, but are not limited to, tissue factor, IL8, urokinase, and tPA.

A standard curve can be generated by using cell populations from which the level of the purified marker is to be determined. For example, standard curves can be generated using cultured endothelial cells, PBLs, or monocytes isolated from human volunteers following stimulation with the angiogenic factor, or vascular endothelial mRNA for the marker protein.

An example of the VEGF-stimulated production of a marker protein (e.g. tissue factor) from monocyte enriched PBLs is illustrated in the following.

Procedure:

1. Collect Whole blood (100-500 mL) from a normal volunteer via venipuncture into vacutainer tubes containing an anticoagulant such as sodium citrate.

2. Centrifuge at 400 g for 15 minutes at room temperature.

3. Collect the buffy layers and dilute to a ratio of 1:3 with normal saline. Layer the diluted buffy layers (30-35 mL) gently into a tube containing 15 mL of Ficoll-Hypaque with a density of 1.077 g/mL. Centrifuge at 400 g for 30 minutes at room temperature.

4. Following centrifugation, collect the cells from the interface by means of a pasteur pipette. Transfer to clean 50 mL polypropylene tubes and add saline to wash cells and remove the Ficoll-Hypaque.

5. Centrifuge cells at 1000 g at room temperature for 6 minutes. Aspirate off supernatant and discard. Resuspend cells in serum free media and plate onto 6 well plates (approximately one plate per 100 mL whole blood starting material). Allow monocytes to adhere for 30 minutes.

6. Following incubation, gently swirl the plates to lift off non-adherent cells. Gently remove non-adherent cells with a pasteur pipette and wash adhered cells with serum.

7. Following washing, pre-incubate adherent cells in the presence or absence of various concentrations of Flk-1 antagonist. The length of the incubation period will vary depending on the pre-determined ability of the antagonist of interest to penetrate cell membranes and the clinical dosing regimen and the pharmacokinetics properties of the antagonist of interest.

8. Following pre-incubation, saturating levels of VEGF (10-100 ng/mL) will be added to the incubation mixtures. The level of VEGF required to maximally stimulate the production of a particular marker of interest will vary depending on the sensitivity of the target cell population used and the turnover of the marker of interest in the cell population used.

9. For quantification of the production of a particular marker protein, cells may be stimulated with VEGF for 6-24 hours. For quantification of the production of mRNA of a particular marker, cells may be stimulated with VEGF for 1-24 hours.

10. Following incubation, supernatants are removed and frozen for later quantification of the production of marker protein if applicable. The adherent cells are lysed by the addition of detergents to generate lysates or the addition of RNAzol to generate mRNA.

11. The levels of marker proteins present on the cell lysates are quantified as described in Example 2. Alternatively, the levels of mRNA present can be quantified using RT-PCR using an internal standard.

12. Following the quantification of samples for the marker protein, a standard curve depicting the absolute levels of marker protein following stimulation with saturating levels of VEGF vs. the concentration of antagonist in the pre-incubation mixture can be generated.

13. Alternatively, the % inhibition of production of marker protein following stimulation with saturating levels of VEGF in the presence of varying concentrations of drug compared to that in the absence of drug can be plotted against the concentration of the antagonist in the pre-incubation mixture to generate a standard curve.

14. Parallel standard curves can be generated using standardized levels of mRNA instead of the levels of marker protein.

15. The % inhibition of a marker protein of interest can be determined by quantifying the levels in cancer patient samples obtained before and after treatment with antagonist in the clinic as specified in the clinical protocol.

16. Using the standard curve generated with parallel samples from normal human volunteers, the levels of antagonist that the cells from the patients might have been exposed to can be estimated. This will be referred to the level of antagonist on target and the efficacious dose of the antagonist.

17. The efficacious level of antagonist on target can be compared to actual levels of antagonist measured in the plasma of the patients at the time of the blood draw post antagonist treatment in clinic. This will allow for the determination of the differences of the “efficacious level” of the antagonist vs. the circulating levels of antagonist in the patients, if any.

One skilled in the art would readily appreciate that the present invention is well adapted to carry out the objects and obtain the ends and advantages mentioned, as well as those inherent therein. The molecular complexes and the methods, procedures, treatments, molecules, specific compounds described herein are presently representative of preferred embodiments and are exemplary and are not intended as limitations on the scope of the invention. Changes therein and other uses will occur to those skilled in the art which are encompassed within the spirit of the invention as defined by the scope of the claims.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are incorporated herein by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that the use of such terms and expressions indicates the exclusion of equivalents of the features shown and described or portions thereof. It is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are within the following claims.

What is claimed is:

1. A method of determining an efficacious dose of a compound administered to a subject for the purpose of modulating angiogenesis, comprising the step of
(a) administering the compound to a patient;
(b) monitoring a marker related to angiogenesis;
(c) constructing a standard curve; and
(d) determining the efficacious dose based on the standard curve.

2. The method of claim 1, wherein said angiogenesis is modulated to treat or prevent conditions associated with angiogenesis including conditions manifested by cell proliferation, cell differentiation, or cell survival.

3. The method of claim 2, wherein said conditions associated with cell proliferation is selected from the group consisting of cancer, arthritis, endometriosis, and ocular neovascularization.

4. The method of claim 1, wherein said compound is selected from the group consisting of a receptor agonist and a receptor antagonist.

5. The method of claim 4, wherein said compound is a receptor antagonist that inhibits a receptor involved in angiogenesis.

6. The method of claim 5, wherein said receptor involved in angiogenesis is selected from the group consisting of Flt-1 and Flk-1.

7. The method of claim 5, wherein said drug is an indolinone compound, having the structure set forth in formula I: 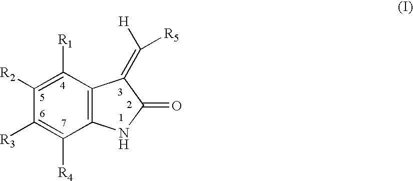
wherein
(a) R₁, R₂, R₃, and R₄ are selected from the group consisting of hydrogen, trihalomethyl, hydroxyl, amine, thioether, cyano, alkoxy, alkyl, amino, bromo, fluoro, chloro, iodo, mercapto, thio, cyanoamido, alkylthio, aryl, heteroaryl, carboxyl, ester, oxo, alkoxycarbonyl, alkenyl, alkoxy, nitro, alkoxyl, and amido moieties; and
(b) R₅ is an optionally substituted aryl or heteroaryl cyclic moiety;
or a pharmaceutically acceptable salt, ester, amide, prodrug, isomer, and metabolite thereof.

8. The method of claim 7, wherein said indolinone compound is selected from the group consisting of

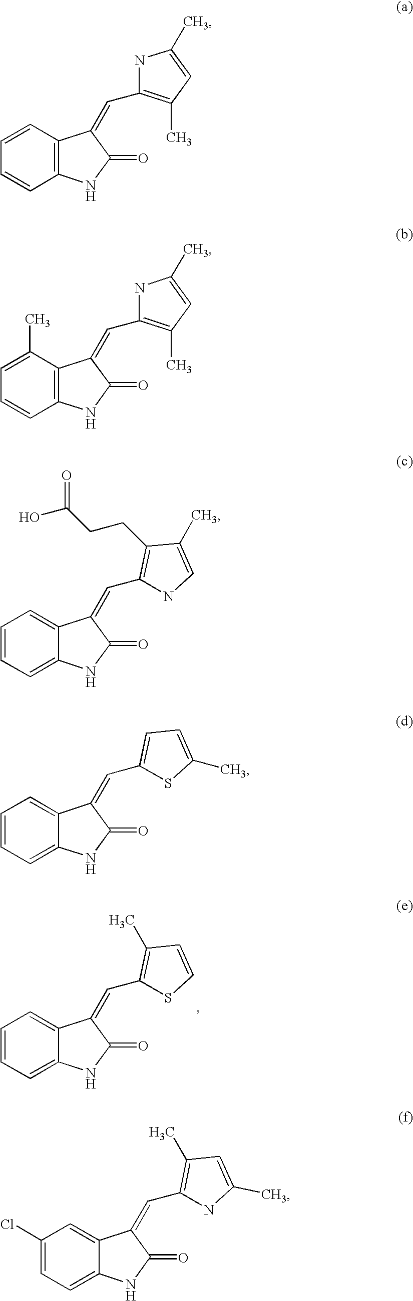

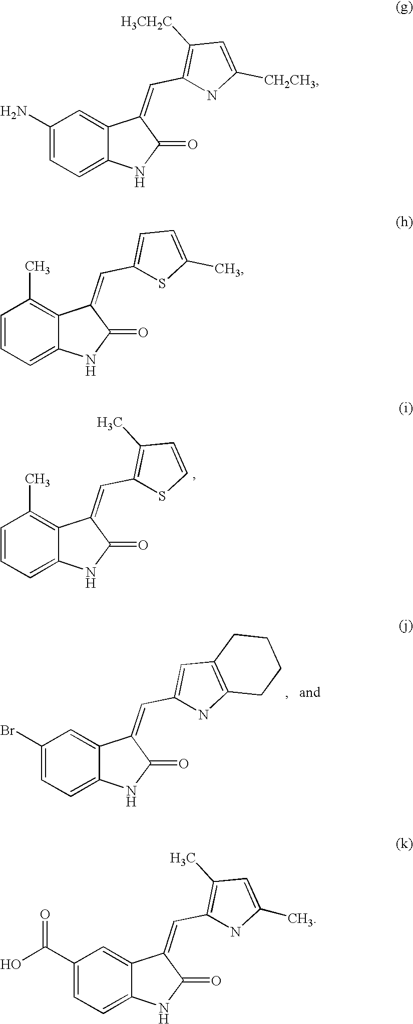

9. The method of claim 1, wherein said marker is present in a sample obtained from said subject.

10. The method of claim 9, wherein said sample is selected from the group consisting of whole blood, a blood fraction, blood plasma, blood serum, cells isolated from blood, whole urine, a urine fraction, saliva, cells isolated from saliva, spinal fluid, amniotic fluids, and biopsy of endothelial cells.

11. The method of claim 10, wherein said sample comprises monocytes.

12. The method of claim 11, comprising the step of adding a receptor agonist to said sample.

13. The method of claim 12, wherein said receptor agonist enhances angiogenesis.

14. The method of claim 13, wherein said receptor agonist is vascular endothelial cell growth factor.

15. The method of claim 1, wherein said marker is selected from the group consisting of cell division, cell motility, cell proliferation, cell death, cell survival, cell differentiation, protein phosphorylation, protein expression, protein glycosylation, mRNA expression, cellular membrane potential, DNA division, DNA methylation, and post-translational modification of a protein.

16. The method of claim 1, wherein said marker is selected from the group consisting of DNA, RNA, mRNA, and protein.

17. The method of claim 1, wherein said step of monitoring a marker comprises the step of determining the presence or amount of said marker.

18. The method of claim 17, wherein said presence or amount of said marker is detected using an antibody.

19. The method of claim 17, wherein said presence or amount of said marker is determined by measuring blood clotting.

20. The method of claim 1, wherein said step of monitoring a marker related to angiogenesis comprises the step of determining the presence or amount of marker mRNA.

21. The method of claim 20, wherein said presence of marker mRNA is determined using reverse transcriptase polymerase chain reaction or a polynucleotide probe.

22. The method of any one of claims 1 and 15-21, wherein said marker is selected from the group consisting of tissue factor, CD40, u-PA, ETS-1, IL8, and t-PA.

23. The method of any one of claims 15-21, wherein said marker is present in a sample obtained from a subject.

24. The method of claim 1, wherein said step of monitoring a marker related to angiogenesis comprises the step of comparing said marker to a standard.

25. A method of treating a disease using the efficacious dose of a drug determined by the method of claim 1.

26. The method of claim 25, wherein said disease is related to angiogenesis.
